(19) United States
(12) Patent Application Publication
(10) Pub. No.: US 2009/0022801 A1
(43) Pub. Date: Jan. 22, 2009

(54) Compositions and Methods for Promoting the Healing of Tissue of Multicellular Organisms

(76) Inventor: David Vachon, Spokane, WA (US)

Correspondence Address: WELLS ST. JOHN P.S., 601 W. FIRST AVENUE, SUITE 1300, SPOKANE, WA, 99201, US

(21) Appl. No.: 12/162,990

(22) PCT Filed: Jan. 31, 2007
(86) PCT No.: PCT/US07/02780

§ 371 Date: Jul. 31, 2008

Related U.S. Application Data
(60) Provisional application No. 60/764,033, filed on Jan. 31, 2006.

(51) Int. Cl. A61K 9/00; A61K 38/00; A61P 29/00; A61P 35/04; A61K 31/74

(52) U.S. Cl. 424/484; 514/2; 424/78.08; 424/488; 424/486

(57) ABSTRACT

Methods are provided for promoting the healing of tissue of a multicellular organism. The methods can include administering a therapeutically effective amount of a polysulfonated material in a liquid mixture to reduce one or both of inflammation and cancerous cell growth. The methods may alternatively or additionally include internally administering a therapeutically effective amount of a polysulfonated material associated with a solid material to reduce one or both of inflammation and cancerous cell growth. Compositions for healing the tissue of a multicellular organism are provided that can include a sulfonated material in a liquid mixture, as well as solid particles.

### RELATED APPLICATION

This application is related to U.S. Provisional Patent Application Ser. No. 60/764,033 entitled “Method For The Reduction of Protease Levels and Delivering Cationic Therapeutic Agents Using Water-Soluble Polyanionic Oligomers & Polymers & Their Salts” filed Jan. 31, 2006.

### TECHNICAL FIELD

Compositions and methods for promoting the healing of tissue of multicellular organisms.

### BACKGROUND

The biochemical environment of the nonhealing wound (as well as serious wounds and/or chronic wounds) is different from that of the normal healing wound in ways that negatively affect multiple aspects of the healing process.

Wounds heal by utilizing a combination of three mechanisms. In each wound, one of the three mechanisms can predominate. The three mechanisms of wound healing are contraction, epithelialization, and connective tissue deposition. Contraction is the method by which wound healing occurs at an amputation site such as the tip of a finger. Epithelialization can predominate in the healing of abrasions and connective tissue deposition occurs when lacerations are sutured closed. The stages of healing are hemostasis, inflammation, proliferation and remodeling. In each of these stages, specific components can play a part through several mediators. In hemostasis, platelets, endothelial cells, fibrin and fibronectin act through growth factors and cytokines. Cytokines are non-antibody proteins that are released from some cells and act as intracellular mediators. Cytokines include lymphokines and interleukins. Inflammation occurs through the action of neutrophils, macrophages and lymphocytes mediated by growth factors and proteases. Proliferation takes place through the actions of fibroblasts, epithelial and endothelial cells and is largely dependent on growth factors and collagen deposition. Remodeling is characterized by collagen cross linking and collagen degradation increasing scar strength as maturation of scar formation occurs.

Normal wound healing can be considered a balance of damaged tissue removal and new tissue formation. Many processes are present that can regulate the biological processes and pathways associated with normal wound repair. An alteration in any of these physiological processes can lead to the formation of a chronic wound.

Inflammation and/or innate immunity, are related to cancerous cell growth. Early in the neoplastic process, inflammatory cells and their released molecular species influence the growth, migration and differentiation of all cell types in the tumor microenvironment, whereas later in the tumorigenic process, neoplastic cells also divert inflammatory mechanisms, such as proteinase production, and chemokine/cytokine functions, in favor of tumor spreading and metastasis. Human polymorphonuclear neutrophils (PMN) comprise 50-70% of circulating leukocytes and induce inflammatory reactions that can be either cytotoxic for tumor cells or aid in tumor growth and metastasis.

The present disclosure provides compositions and methods using the compositions that can reduce one or both of inflammation and cancerous cell growth in multicellular organisms.

### SUMMARY

Methods are provided for promoting the healing of tissue of a multicellular organism. The methods can include administering a therapeutically effective amount of a polysulfonated material in a liquid mixture to reduce one or both of inflammation and cancerous cell growth.

Methods are also provided for promoting healing of tissue of a vertebrate organism. The methods can include internally administering a therapeutically effective amount of a polysulfonated material associated with a solid material to reduce one or both of inflammation and cancerous cell growth.

Compositions for healing the tissue of a multicellular organism are provided that can include a sulfonated material in a liquid mixture. The composition can be configured to be administered to reduce one or both of inflammation and cancerous cell growth.

Compositions for healing the tissue of a multicellular organism are also provided that can include solid particles. The particles can include polysulfonated material, and can be configured to be administered to reduce one or both of inflammation and cancerous cell growth.

### FIGURES

FIG. 1 is an example depiction of the interaction of compositions of the disclosure with enzymes produced by neutrophils, according to an embodiment of the disclosure.

FIG. 2 is an example depiction of the interaction of compositions of the disclosure with tissue fluids including salts, and enzymes produced by neutrophils, according to an embodiment of the disclosure.

FIG. 3 are example preparations of compositions of the disclosure according to an embodiment of the disclosure.

FIG. 4 are example preparations of compositions of the disclosure according to an embodiment of the disclosure.

FIG. 5 is a depiction of an example application according to an embodiment of the disclosure.

FIG. 6 is a depiction of an example application according to an embodiment of the disclosure.

### DESCRIPTION

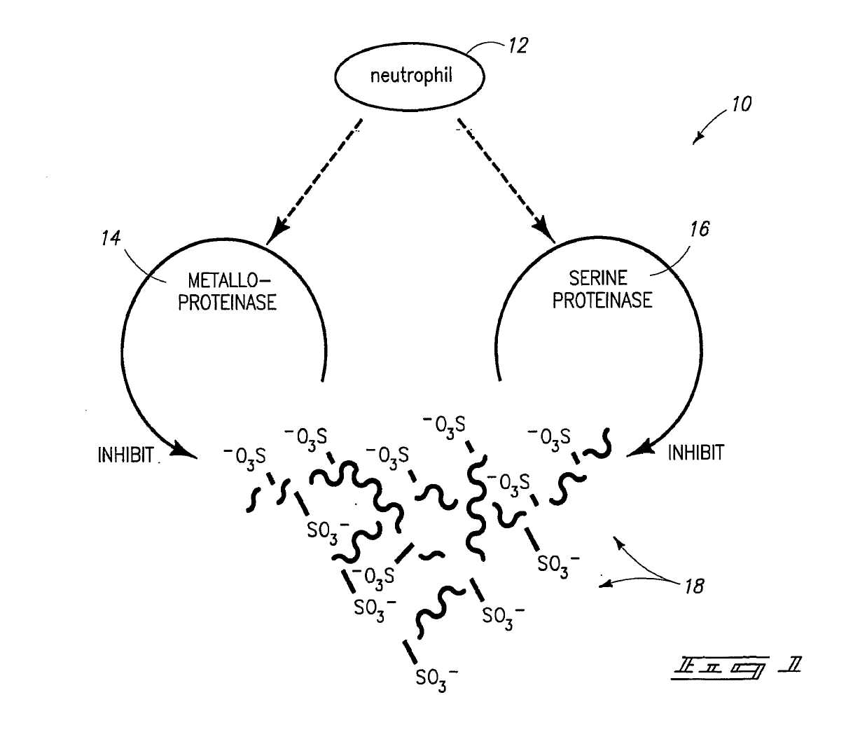

Compositions and methods of the present disclosure will be described with reference to FIGS. 1-6. Referring to FIG. 1, a general proteinase inhibition scheme 10 is shown that includes a neutrophil 12 producing a metallo-proteinase 14 and a serine proteinase 16. Scheme 10 further includes the inhibition of proteinases 14 and 16 by a polysulfonated material 18.

Polysulfonated material 18 can be represented chemically as R(SO₃⁻)ₙ, with n being greater than 1 and the R group containing carbon. It is understood that material 18 can be associated with counter ions, such as cations. While these counter ions are not shown in FIG. 1, it is understood that they can be present. The R group can be the backbone of an oligomer, such as a dimer and or trimer, or a polymer for example. In accordance with other implementations, the oligomer or polymer can include monomers such as arylenevinyl sulfonate, styrene sulfonate, sulfated saccharides, and/or vinyl sulfonate monomers as well as nonsulfonated monomers. The oligomer can include repeating units of the same monomer, or more than one monomer.

According to an example implementation, the oligomer can be incorporated into other materials. For example, material 18 can also be a polymer comprising the oligomer. The oligomer can be copolymerized with other monomers and/or other oligomers to form a copolymer. In accordance with embodiments of the disclosure, the polymer can include repeating oligomer units, such as polymers of repeating oligomer units. The oligomer units may be identical monomer units or mixed monomer units. For example, material 18 can be polyarylenevinylsulfonate, polystyrene-sulfonate, polyvinylsulfonate, polyantholesulfonate, and/or acrylamidomethyl propane sulfonate polymer.

Material 18 can also include other sulfonated compounds such as, but not limited to, polymers of sulfated saccharides or polysulfated polysaccharides, such as dextrin sulfate, dextran sulfate, chitosan sulfate, or cellulose sulfate. The sulfonate group of polysulfonated material 18 can be coupled to an —OR, with the R representing the remainder of polysulfonated material 18, and the coupling with the O forming a sulfate group. Accordingly, sulfate groups contain sulfonate groups. Accordingly, material 18 can include polysulfonates including sulfonic acids, sulfonic acid salts, and polysulfated compounds. The polysulfated compounds can include synthetic, semi-synthetic, & naturally occurring polysulfated polysaccharides that include dextran sulfate given as an example above, as well as the sulfated semisynthetic polysaccharide pentosan polysulfate, for example.

Material 18 can have a molecular weight of from about 600 grams/mole to about 1,000,000 grams/mole. As an example, material 18 can be polymer or copolymer having a molecular weight of at least about 70,000 grams/mole. Material 18 can also be water soluble.

Material 18 can also include polysulfonated material blended with another material. For example, polysulfonated material such as polystyrenesulfonate can be blended with materials such as hydrogel(s). Hydrogels can include, but are not limited to, alginates, polyacrylates, polyalkylene oxides, and/or poly (N-vinyl pyrrolidone). The hydrogel may also be amorphous. Material 18 can also be blended with polyurethanes, for example. Material 18 can also be blended with naturally occurring polymers that include chitosan, hyaluronic acid, and starch.

The SO₃⁻ group can be referred to as a sulfonate group. The sulfonate group can be a terminal sulfonate group, and material 18 can include at least one terminal sulfonate group. In accordance with embodiments of the disclosure, the SO₃⁻ groups of the polysulfonated material can extend from the oligomer backbone, such as a polymer or copolymer backbone.

The sulfonate group can take the form of an acid, for example. As an acid, the sulfonate group can be protonated, such as SO₃H. Material 18 can include many sulfonate groups and these sulfonate groups may all be protonated or some may be protonated while others are unprotonated.

According to another embodiment of the disclosure, the sulfonate groups of material 18 may be a component of a salt, such as a metal or organic salt. According to embodiments of this configuration, material 18 can be referred to as a polyanionic salt, such as polymetallosulfonate and/or a polyorganosulfonate. The sulfonate group of material 18 can be associated with either or both of an inorganic or organic element or compound.

In accordance with an implementation, the sulfonate group can be associated with a complimentary cation. As an example, the sulfonate group can be associated with an inorganic species such as one or more of a positively charged Na, Ag, K, Li, Au, Ca, Zn, Mn, Mg, Fe, and/or Ce, such as Na⁺, Ag⁺, K⁺, Li⁺, Au⁺, Ca⁺⁺, Zn⁺⁺, Mn⁺⁺, Mg⁺⁺, Fe++/Fe⁺⁺⁺, and/or Ce⁺⁺⁺. The sulfonate can also be associated with NH₄⁺, for example. According to another example, the sulfonate group can be associated with one or more of organic species including nitrogen containing organic species such as, an amino acid, a tetracycline, doxycycline, arginine, lysine, glutathione, lidocaine, albuterol, and/or alkyl/benzylammonium.

In accordance with an example implementation, material 18 can be sodium polystyrene sulfonate, a neutralized derivative of the corresponding polystyrene sulfonic acid. This polymetallosulfonate may be further exchanged with any variety of metal cations to prepare mono, di, tri, and even tetravalent metal salt derivatives. Similarly, poly(metallo)sulfonate such as sodium polystyrene sulfonate, may be converted to a poly(organo)sulfonate derivative by exchange of sodium for any nitrogen atom containing salt/protonatable nitrogen compound of interest. Example nitrogen atom containing salts/protonatable nitrogen compounds can include, but are not limited to, amines, amidines, imines, thiazoles, imidazoles, and/or pyridines. Additionally, ammonium salt derivatives may be prepared by the exposure of an amino compound to the acid form polysulfonate.

In accordance an embodiment of the disclosure, derivatives of material 18 can be produced by chemically or biochemically modifying material 18. As examples: the cation of material 18 can be modified (substitute Ag⁺ for sodium (Na⁺)); a tetracycline:H⁺ can be substituted for sodium of material 18 via ion exchange; the sulfonic acid derivative of material 18 may be used as a proton source during an acid-base reaction by treatment with, for example, an amino acid, such as an arginine; a biogenic amino such as tyramine or dopamine. Similarly, the polyanion or polysulfonic acid of material 18 can be exchanged with a polycation or polyamine, such as a strongly basic ion exchange resin, for example, poly-L-lysine.

Material 18 can be associated with numerous elements and compounds. For example, material 18 can be associated with paramagnetic ions such as Mn⁺; Gd⁺²Fe⁺³; as well as radio-opaque metal ions of barium; tungsten; and radioactive ions of strontium; rhenium; yttrium; divalent metal cations Ca⁺²; Zn⁺²; Cu⁺²; Mg⁺²; monovalent metal cations Na⁺; Ag⁺; Li⁺; K⁺.

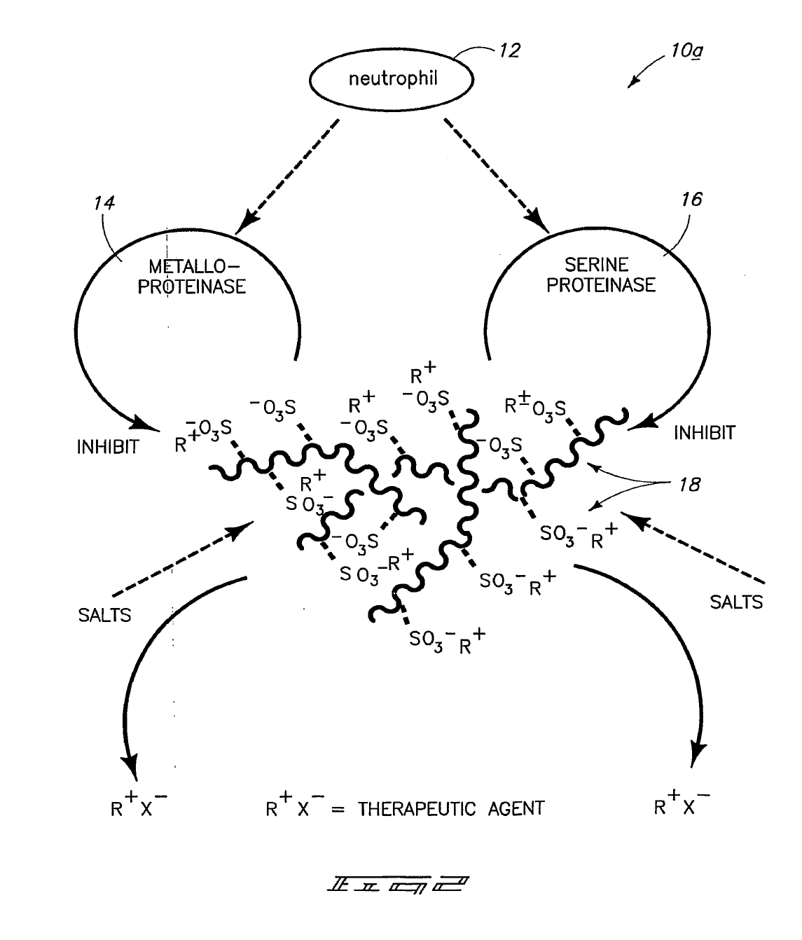

Referring to FIG. 2, scheme 10A is shown with material 18 being associated with at least a portion therapeutic agent R⁺. Example agents R⁺ associated and/or coupled to material 18 are provided herein. When provided to inhibit inflammation or cancerous cell growth, the portion of therapeutic agent R⁺ can be released from material 18 and form therapeutic agent R⁺X⁻, via ionic exchange, for example. According to an implementation, material 18 can simultaneously provide both proteinase inhibition as well as therapeutic agent.

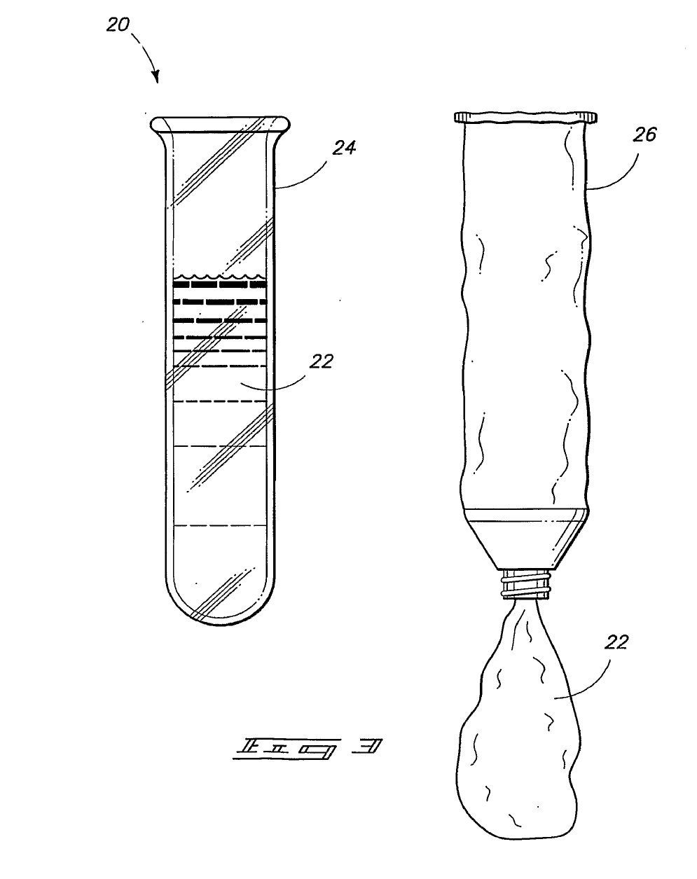

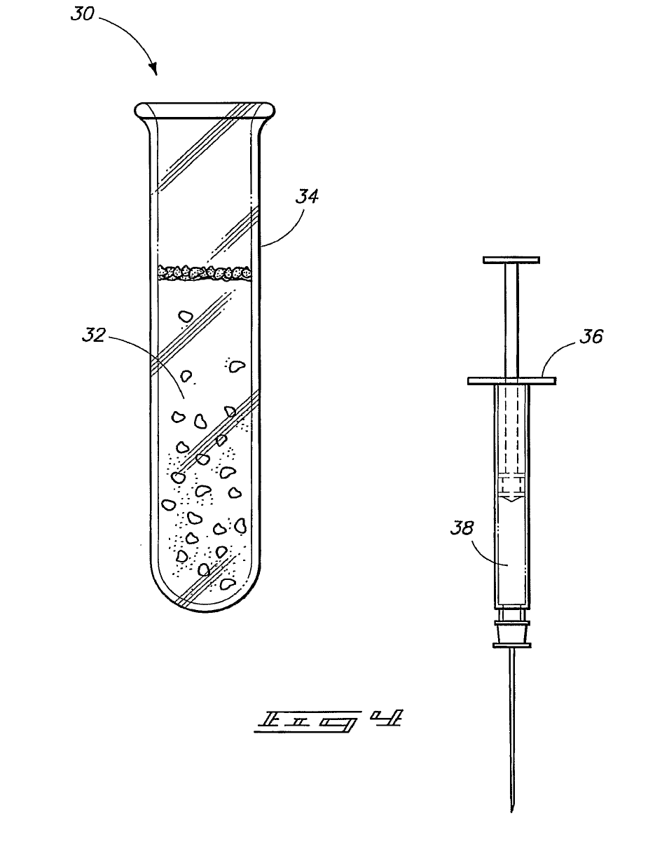

Referring to FIGS. 3 and 4, preparations of material 18 are shown in both liquid (FIG. 3) and solid (FIG. 4) form. Referring to FIG. 3, preparation 20 includes a mixture 22 within container 24. Mixture 22 can include at least two components with at least one of the two components being material 18. According to an embodiment of the disclosure, mixture 22 can be a liquid mixture. Material 18 can be present in mixture 22 in the form of a soluble component, for example, or in the form of an insoluble component, as another example. Mixture 22 can be hydrophilic such as water, for example, and material 18 can be water soluble. Mixture 22 can also be hydrophobic, such as an oil or fatty acid, and material 18 can be formulated to be hydrophobic as well.

In accordance with an example implementation, mixture 22 can include water and material 18, with material 18 being a polystyrene sulfonate. Mixture 18 may be buffered to a pH of from about 3.5 to about 8.0 as required to keep the sulfonate groups of material 18 in the desired configuration.

In accordance with another example implementation, mixture 22 can be homogeneous or heterogeneous. For example, mixture 22 can be a homogeneous mixture of water and water soluble polysulfonated material. As another example, mixture 22 can be a heterogeneous mixture, such as an emulsion. Mixture 22 can include a gel, cream, or lotion, for example. Mixture 22 can include carboxymethylcellulose, for example. Mixture 22 can include additional components as well as material 18. The additional components may include but are not limited to detergents, excipients, wetting agents, and skin permeation enhancers. The detergents can include tween 80 (Polysorbate 80), for example. The skin permeation enhancers can include one or more of a linoleic acid, an alpha-linoleic acid, an oleic acid, cod liver oil, menthol derivatives, squalene, glycerol derivatives, herbal ingredients, and senkyu ether extract. Mixture 22 can be a neutral, hydrophilic matrix cream, lotion or gel, and material 18 can be solubilized or dispersed into this mixture. The gel, for example, can include any variety of largely aqueous based formulations that include, but are not limited to, a hydrophilic water-soluble polymer, a humectant, a preservative, and/or purified water.

Material 18 may be associated and/or provided with natural and/or synthetic peptides. Material 18 may also be associated with and/or provided in a preparation with: methotrexate; fluorouracil; adriamycin; ansamitocin; cytosine arabinoside; arabinosyl adenine; mercaptopolylysine; PAM; L-PAM; phenylalanine mustard); mercaptopurine; mitotane; procarbazine dactinomycin (actinomycin D); mitomycin; plicamycin (mithramycin); aminoglutethimide; estramustine; flutamide; leuprolide; megestrol; tamoxifen; amsacrine (m-AMSA); asparaginase (L-asparaginase) Erwina asparaginase; etoposide (VP-16); interferon .alpha.-2a; interferon .alpha.-2b, teniposide (VM-26); adriamycin; arabinosyl; procarbazine; and dacarbazine. In accordance with additional embodiments of the disclosure, material 18 may also associated with and/or provided in a preparation with: Nitrogen mustards: (Chlorambucil, Chlormethine, Cyclophosphamide, Ifosfamide, Melphalan). Nitrosoureas: (Carmustine, Fotemustine, Lomustine, Streptozocin). Platinum: (Carboplatin, Cisplatin, Oxaliplatin, BBR3464). Busulfan, Dacarbazine, Mechlorethamine, Procarbazine, Temozolomide, ThioTEPA, Uramustine; Antimetabolites: Folic acid: (Methotrexate, Pemetrexed, Raltitrexed). Purine: (Cladribine, Clofarabine, Fludarabine, Mercaptopurine, Thioguanine). Pyrimidine: (Capecitabine). Cytarabine, Fluorouracil, Gemcitabine; Vincaalkaloids: (Vinblastine, Vincristine, Vindesine, Vinorelbine); Cytotoxic/antitumor antibiotics: Anthracycline family: (Daunorubicin, Doxorubicin, Epirubicin, Idarubicin, Mitoxantrone, Valrubicin). Bleomycin, Mitomycin; Topoisomerase inhibitors: Topotecan, Irinotecan; Monoclonal antibodies: Alemtuzumab, Bevacizumab, Cetuximab, Gemtuzumab, Panitumumab, Rituximab, Infliximab, Tositumomab, Trastuzumab, Etanercept; Photosensitizers: Aminolevulinic acid, Methyl aminolevulinate, Porfimer sodium, Verteporfin; Kinase Inhibitors: Dasatinib, Erlotinib, Gefitinib, Imatinib, Lapatinib, Nilotinib, Sorafenib, Sunitinib, Vandetanib (ZD6474).

Additional compounds that may be provided and/or associated with material 18, include: Altretamine, Anagrelide, Bortezomib, Denileukin diftitox, Estramustine, Pentostatin, Pegaspargase, Alagebrium (3-phenacyl-4,5-dimethylthiazolium, anti-helmintics; antitoxins; antivenins; aminoglycosides; theophylline; aminophylline; hemin; hematoporphyrins; muramyldipeptide; muramyltripeptide; lymphokines; macrophage activation factor; N-acetyl-muramyl-L-alanyl-D-isoglutamine; ketoconazole; nystatin; griseofulvin; flucytosine (5-fc); miconazole; amphotericin B; ricin; cyclosporins; sulfazecin; growth hormone, melanocyte stimulating hormone; triamcinolone; fludrocortisone; oxytocin; vassopressin; cyanocobalamin; super oxide dismutase; alkaline phosphatase; amelexanox; glutathione; carnosine; p-aminosalicylic acid; isoniazid; capreomycin; cycloserine; ethambutol; ethionamide; pyrazinamide; rifampin; and streptomycin; acyclovir; amantadine azidothymidine; ribavirin and vidarabine; diltiazem; nifedipine; verapamil; dapsone; chloramphenicol; neomycin; cefaclor; cefadroxil; cephalexin; erythromycin; clindamycin; lincomycin; bacampicillin; carbenicillin; dicloxacillin; cyclacillin; picloxacillin; hetacillin; methicillin; nafcillin; oxacillin; penicillins (G&V); ticarcillin; rifampin; doxycycline; mefenamic acid; oxyphenbutazone; phenylbutazone; piroxicam; sulindac; tolmetin; chloroquine; hydroxychloroquine; metronidazole; quinine; quinidine; meglumine; penicillamine; paregoric; codeine; heroin; methadone; morphine; opium; and papaverine; noscapine; deslanoside; atracuriurm; gallamine; metocurine; pancuronium; succinylcholine (suxamethonium); tubocurarine; vecuronium; ethchlorvynol; flurazepam; glutethimide; methotrimeprazine; methyprylon; midazolam; temazepam; triazolam; bupivacaine; chloroprocaine; etidocaine; lidocaine; mepivacaine; procaine; marcaine; tetracaine; droperidol; etomidate; fentanyl; ketamine; benzyl trimethyl ammonium, chlorhexidine; amino acids (natural & synthetic); nicotinic acid; nicotinamide, pyridoxine; nucleosides (purines); thiamine; coenzyme A; pentoxifylline; 3-amino-4-hydroxybutyric acid; 6-diazo-5-oxo-L-norleucine; aceclofenac; acediasulfone; alminoprofen; amfenac; amoxicillin; ampicillin; apalcillin; apicycline; aspoxicillin; azaserine; aztreonam; bambermycin(s); biapenem; bromfenac; bucillamine; bumadizbn; candicidin(s); carbenicillin; carprofen; carumonam; carzinophillin A; cefamandole; cefatrizine; cefbuperazone; cefclidin; cefdinir; cefditoren; cefepime; cefetamet; cefixime; cefmenoxime; cefminox; cefodizime; cefonicid; cefoperazone; ceforamide; cefotaxime; cefotetan; cefotiam; cefozopran; cefpimizole; cefpiramide; cefpirome; cefprozil; cefroxadine; ceftazidime; cefteram; ceftibuten; ceftriaxone; cefuzonam; cephaloglycin; cephalosporin C; cephradine; ciprofloxacin; clinafloxacin; cyclacillin; denopterin; diclofenac; edatrexate; enfenamic acid; enoxacin; epicillin; etodolac; flomoxef; flufenamic acid; grepafloxacin; hetacillin; imipenem; lomefloxacin; lymecycline; meclofenamic acid; melphalan; meropenem; moxalactam; mupirocin; mycophenolic acid; nadifloxacin; niflumic acid; norfloxacin; oxaceprol; panipenem; pazufloxacin; penicillin N; pipemidic acid; podophyllinic acid 2-ethylhydrazide; procodazole; pseudoephedrine; pteropterin; quinacillin; ritipenem; romurtide; S-adenosyl, methionine; salazosulfadimidine; sparfloxacin; streptonigrin; succisulfone; sulfachrysoidine; sulfaloxic acid; teicoplanin; temafloxacin; temocillin; tetracycline; tolfenamic acid; (N-((5-(((1;4-Dihydro-2-methyl-4-oxo-6-quinazolinyl)methyl)methylamino)-2-thienyl)carbonyl)-L-glutamic acid); tosufloxacin; trovafloxacin; doxyxycline; mafenide; minicycline; tigemonam; or vancomycin; lucensomycin; natamycin or; 6-diazo-5-oxo-L-norleucine; denopterin; edatrexate; eflomithine; (N-((5-(((1;4-Dihydro-2-methyl-4-oxo-6-quinazolinyl)methyl)methylamino)-2-thienyl)carbonyl)-L-glutamic acid)-ubenimex. In accordance with yet another example, material 18 can be associated and/or provided with albuterol, terbutaline, and/or ephedrines.

Mixture 22 can be provided to an application apparatus such as application apparatus 26. In the depicted embodiment, apparatus 26 is a collapsible tube. Mixture 22 can take form of a lotion or gel which can be extruded, from apparatus 26 upon application of force. In accordance with another embodiment, mixture 22 can be provided to a container configured for pressurization such as an aerosol can or an inhaler. In one implementation, mixture 22 can include a propellant and material 18. Under pressurization, mixture 22 can be expelled from the pressurized container in aerosol form. Mixture 22 may be provided from a nebulizer or inhaler as well.

Referring to FIG. 4, preparation 30 is shown that includes particles 32 within container 34. Particles 32 can be solid and include polysulfonated material 18, for example. In accordance with an example configuration, individual ones of particles 32 can be hydrogel beads. The hydrogel of the hydrogel beads can be cross-linked in the presence of and/or blended with to include material 18 to form a solid blend. The hydrogel can polyethylene glycol-based and/or polyvinyl alcohol-based, for example. In accordance with other embodiments of the disclosure, material 18 can be dispersed into a solid matrix of cross-linked acrylic acid-based polymer such as methacrylic acid or any of its esters including poly(2-hydroxy ethyl methacrylate) (HEMA), for example, polypropylene oxide, polyethylene oxide, polyvinyl alcohol, polyurethane, alginate, silicone, hydrocolloid, and/or the hydrogel. Further, individual ones of particles 32 can include poly(N-vinyl pyrrolidone), poly(vinyl alcohol), poly(acrylic acid), polyacrylamide including poly(N-isopropylacrylamide), poly(ethylene-co-vinyl acetate), poly(ethylene glycol)/polyethylene oxide, poly(methacrylic acid), polyurethanes, and silicones.

In accordance with another implementation, individual ones of particles 32 can include material 18 as a biodegradable polymer or material 18 associated with a biodegradable polymer. Example biodegradable polymers include, but are not limited to, lactide/glycolides, polyglycolides, polyorthoesters, and/or polylactides.

Individual ones of the particles can be microspheres that include material 18. In accordance with another implementation, individual ones of particles 32 can include a degradable substrate, such as collagen, for example. Individual ones of particles 32 can also include gelatin or the heterosaccharide pectin.

As an example, apparatus 36 can be used to apply particles 32. An example of apparatus 36 includes a syringe; however additional applicators may be utilized, such as gauze and/or collapsible tubing. In accordance with an example embodiment, particles 32 may be provided to apparatus 36 in the form of an injectable mixture. Particles 32 within the injectable mixture may or may not be dissolved. In accordance with another implementation, particles 32 can be material 18 of mixture 22. As a component of mixture 22, particles 32 may be provided as material 18 according to example embodiments.

Referring to FIGS. 3 and 4, preparations 20 and 30, respectively are not mutually exclusive. Compositions that may be included within mixture 22 may also be incorporated into particles 32. Likewise, compositions that may be included within particles 32 may also be incorporated into mixture 22. According to example implementations, preparations 20 and/or 30 may include biologically active material. Example biologically active materials can include, but are not limited to, one or more of peptides, proteins, cytokines, healing factors, antibiotics, cytotoxins, VEGF, PDGF, EGF or other relevant growth factors, including, but not limited to exogenous growth factors. Preparations 20 and/or 30 may also include one or more of an angiogenesis stimulant, antibacterial, antibiotic agent, or antiangiogenic agent. According to an example implementation, material 18 may inhibit the degradation of exogenous and/or endogenous factors. For example, material 18 may be provided with an exogenous material to an organism. Material 18 may prevent the degradation of the exogenous material providing for the therapeutic activity of the exogenous material. Material 18 and the exogenous and/or endogenous materials may be provided simultaneously to the organism.

Preparations 20 and/or 30 may have a concentration of material 18 of about 1 mg/ml, although higher or lower concentrations can be used if desired. For example, concentrations as low as about 0.1 mg/ml, or as high as the limit of solubility of material 18 in mixture 22 and/or particles 32, may be used in a formulation such as amorphous gel or solid dressing such as a those fabricated of calcium alginate. Preparations 20 and/or 30 may contain a concentration of material 18 of from about 1 to about 500 mg/ml.

Preparations 20 and/or 30 may be applied via short or long term application. Preparations including vehicles such as sterile PBS or sterile DI water are suitable for short term application of the inhibitor. For longer term application, use of a slow release vehicle may be utilized. For example, a gel formulation preparation can be used for effective delivery of material 18.

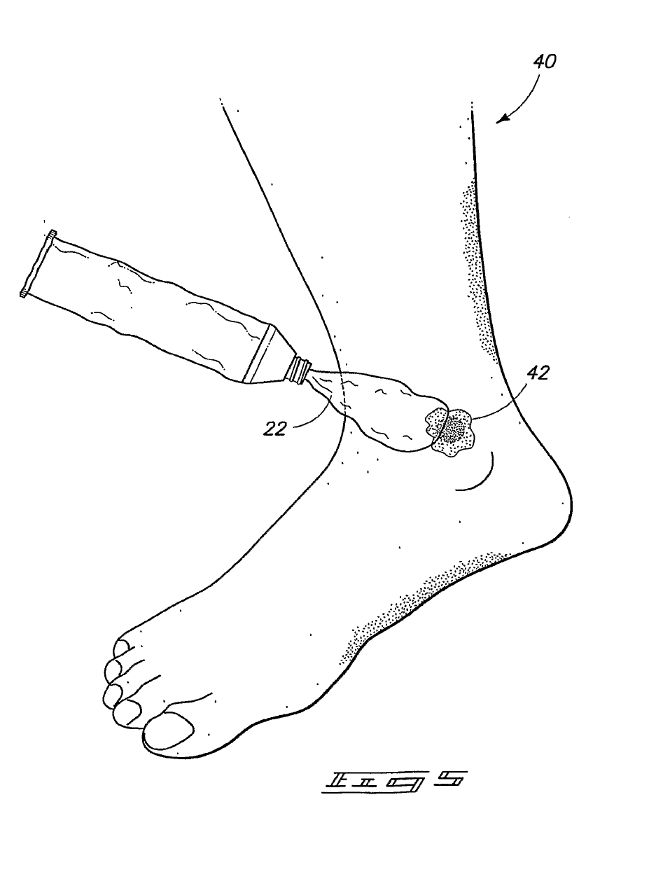

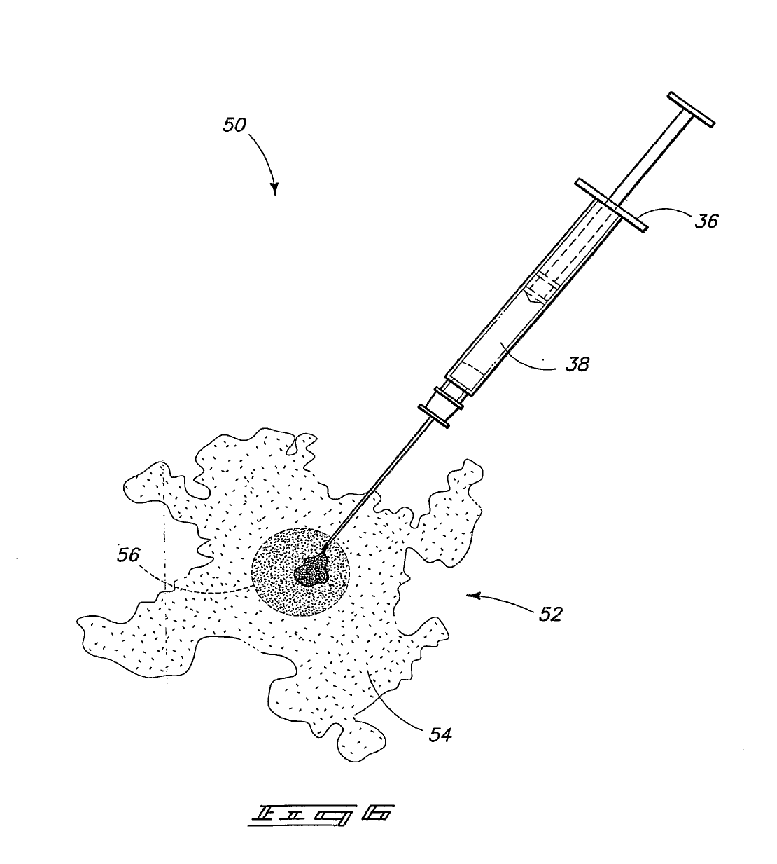

Referring to FIGS. 5 and 6, example methods for applying preparations 20 and 30 are depicted. In accordance with example embodiments, these methods can promote the healing of tissue of a multicellular organism, including but not limited to vertebrate organisms. In accordance with example implementations, a therapeutically effective amount of material 18 can be administered to the organism to reduce one or both of inflammation and cancerous cell growth.

In general, chronic wounds can be characterized by a prolonged inflammatory phase, which ultimately can result in elevated protease activity and the subsequent degradation of growth factors and other positive wound healing factors, with the overall effect being impaired healing. Chronic, wounds can be considered an imbalance between tissue deposition, stimulated by growth factors, and tissue destruction mediated by proteases. Chronic wounds, of diverse etiologies can have elevated levels of a specific class of proteolytic enzymes known as the matrix metalloproteases (MMPs). The effects of these high levels of MMPs in the wound environment may include local destruction of growth factors and their receptors as well as degradation of granulation tissue components.

While the overall goal of wound healing is to synthesize and deposit new tissue so as to reestablish continuity and function, it can be noted that controlled tissue degradation is a normal part of the wound healing process. Much of the tissue degradation required in wound healing is performed by MMPs. The MMPs are a family of structurally related, protein-degrading enzymes that require calcium ions for structural conformation and zinc ions in their active site for function. About 20 different members of the family have been identified, and they share similar structure (about 40% amino acid homology). Multiple cell types, including macrophages, fibroblasts, neutrophils, epithelial cells, and endothelial cells, synthesize MMPs in the presence of specific biochemical signals, such as inflammatory cytokines (e.g., TNF*, IL-1b). MMPs play a role in many normal physiological processes, such as wound healing, embryonic development, and menstruation. An individual MMP may have one or multiple protein substrates that it degrades. Certain MMPs are very specific in their function (e.g., the collagenases only degrade collagen). Specifically, they cleave the collagen triple helix at a single point. This cleavage then allows the rigid triple helix to relax and unravel, resulting in two gelatin fragments. Other MMPs have multiple substrates; some redundancy of substrates between MMPs is evident. When redundancy exists, usually one MMP degrades a particular substrate preferentially.

Collectively, the MMP family of enzymes is capable of digesting almost all of the components of the extracellular matrix. In order for healing to progress and result in repair, a balance may exist between the protein-degrading activities of MMPs and other cellular activity directed towards the synthesis and deposition of the protein components of granulation tissue. The proteolytic activity of MMPs is controlled by various mechanisms, including gene transcription, production of the enzyme in an inactive form (called a zymogen) that requires extracellular activation, and by local secretion of endogenous enzyme inhibitors called tissue inhibitors of metalloproteases (or TIMPs). The same cells that produce MMPs can synthesize TIMPs. Four different TIMPs have been identified in tissues (TIMP-1; TIMP-2, TIMP-3, and TIMP-4). These TIMPs can inhibit all of the MMPs by binding to the zinc-containing active site of the enzyme. TIMPs do not bind to the zymogen form of the enzyme. During normal wound repair, a delicate balance can exist between the MMP and TIMP activity levels. If the balance is disturbed, high levels of MMPs may result in excessive tissue degradation or destruction of other protein components in the extracellular matrix (ECM), such as growth factors, cell surface receptors, and even the TIMPs themselves.

At least one other characteristic of some chronic wounds is the excess of proteases that are detected extracellularly. While controlled degradation can occur during normal wound healing, excess or prolonged proteolytic activity is considered detrimental and thought to contribute to the lag in healing of the wound.

With regard to cancerous cell growth, most of the neutrophil-induced tumor-promoting effects are attributed to their abilities to release proteases. Neutrophil degranulation results in the release of serine proteases, such as elastase, cathepsin G and protease-3, which may contribute to the activation of MMPs that mediate tumor cell invasiveness.

Tumorigenesis involves not only tumor cells that become transformed but also the tumor stroma which reacts by inducing inflammatory and angiogenic responses. Angiogenesis, the formation of new capillaries from preexisting vessels, is typically required for tumor growth and metastasis. During angiogenesis, quiescent endothelial cells are activated and they initiate migration by degrading the basement membranes through the action of specific (expressed) proteases, in particular, MMPS.

MMPs promote tumor progression not only through ECM degradation but also through signaling functions. MMPs counter apoptosis, orchestrate angiogenesis, regulate innate immunity, and promote metastasis and tumor growth. Stromal and immune-defense responses can eventually fail, resulting in immune-cell evasion, phenotypic evolution of metastases, chemotherapeutic resistance and further tumor dissemination. MMP binding to cell-surface proteins may have an effect on intracellular signaling, facilitate proenzyme localization and activation, mediate cell motility by disruption of cell contacts with the ECM, and promote internalization of the enzyme. For example, integrins are shown to act as receptors for several proteases, including MMPs. Such interactions have been detected in caveolae, in invadopodia, and at the leading edge of migrating cells, where directed proteolytic activity is likely to be needed. The first interaction between an integrin and an MMP (MMP-2) was identified on the surface of melanoma cells and angiogenic blood vessels. Furthermore, MT1-MMP was shown to activate the integrin, αVβ3, through proteolytic cleavage. Additionally, αVβ3-integrin may have modulatory properties on MMP-2 activity by binding to its C-terminal domain.

Further, CD44, which is the principal receptor for hyaluronan, can also serve as a MMP-9-docking molecule. Interaction of MMPs with the cell surface not only may be needed for proenzyme activation and targeting at specific sites for degradation of cell-surface substrates, but also could promote intracellular degradation via receptor-mediated endocytosis.

Leukocyte elastase (LE) is a serine protease, expressed by polymorphonuclear (PMN) leukocytes, mainly neutrophils, which acts both at the intra-cellular level to kill engulfed pathogens, and at the extra-cellular level as mediator of coagulation, immune responses, and wound debridement. Since LE has the potential to degrade some structural proteins of the extra-cellular matrix (ECM), such as elastin, fibronectin and collagens, production of excess amounts of active LE has been identified in a number of pathological conditions leading to impairment of ECM organization that include rheumatoid arthritis, emphysema, chronic obstructive pulmonary disease (C.O.P.D.), cystic fibrosis, some chronic wounds, inflammatory bowel diseases, and tumor progression for example. LE also activates the pro-enzymatic form of matrix metalloproteinase-9 (MMP-9) massively released by the PMNs, and instrumental to their extravasation. Human tissues are normally protected from excessive LE activity by endogenous inhibitors such as α1-protease-inhibitor (α1-PI), α2-macroglobulin, and secretory leukoprotease inhibitor (SLPI). An enzyme/inhibitor imbalance may lead to increased lysis of ECM macromolecules and thus an increased risk of tissue injury in areas infiltrated by activated PMNs. Furthermore, given the ability of LE to degrade multiple cytokines, receptors, and complement components, a negative modulation of the inflammatory response may favor antigen persistence, leading to chronic inflammation. As for the possibility of using exogenous LE-inhibitors for therapeutic purposes, to date many of the inhibitors that have been developed present side effects that make them less than ideally suitable for human use. However, material 18 may be provided to the organism in an effort to protect both exogenous and endogenous factors.

Referring to FIG. 5, organism 40 may have a wound 42, such as an epidermal wound. Example wounds include, but are not limited to, burns (thermal and chemical) and chronic ulcers, such as pressure ulcers, diabetic ulcers, venous leg ulcers, and periodontitis. Wound 42 can also include atopic dermatitis, a common form of inflammation of the skin and characterized by elevated tissue levels of Cathepsin G. Atopic dermatitis is a chronic skin disorder characterized by pruritus, dry skin, and excoriation, which may be localized to a few patches or involve large portions of the body. Wound 42 can also be surgical or the result of trauma such as abrasions, skin tears, and/or blisters.

In accordance with the embodiment depicted in FIG. 5, mixture 22 including material 18 can be administered topically to wound 42. As described above, mixture 22 can be a liquid such as a gel, cream, or lotion. In accordance with another implementation, mixture 22 including material 18 can be applied to a substrate such as a gauze or sponge, and the substrate can be applied to wound 42.

Upon administration of material 18, protein degradation of the tissue of organism 40 can be inhibited. In accordance with example implementations, protein degradation can be prevented via the inhibition of proteases including metalloproteinases, such as collagenase and gelatinase. Inhibition of both serine proteases and metalloproteinases can also be accomplished. The serine proteases inhibited can include one or both of elastase and cathepsin G.

Mixture 22 may be applied to wound 42 daily, or more or less frequently as required. A typical daily dosage of material 18 will be 20 mu/g/cm² of the wound or ulcer, although it will be recognized that this amount may be varied, and concentrations of 0.1-2000 mu/g/cm² advantageously may be used. For example, ulcers of long duration (such as one year or longer) may require concentrations of 500 mu/g/cm² applied multiple times per day, such as, for example, 2, 3, or 4 times daily. For ulcers of lesser duration, or those that are responding well to higher doses, the dose may be lowered. For example, the protease inhibitor dose may be lowered sequentially to, for example, 100, 10, 1, or 0.1 mu/g/cm². In addition, the application of the inhibitor may be made less frequently, such as from 4 to 1 times daily.

Referring to FIG. 6, tissue 52 is shown having composition 38 applied thereto. Tissue 52 includes both cancerous cells 56 and non-cancerous cells 54. In accordance with an example embodiment, a therapeutically effective amount of composition 38 including material 18 associated with a solid material, such as a microspheres or bead, can be administered internally to reduce one or both of inflammation and cancerous cell growth.

Examples embodiments of the disclosure are provided below.

### EXAMPLE 1

### Preparation of Material 18

Polystyrene sodium sulfonated (PSS, 70,000 mw) acquired from Sigma-Aldrich, PO Box 14508, St. Louis, Mo. 63178, UNITED STATES can be dissolved into Di water to yield a 10% solids solution. Separately, 25 g of Amberlite (Purolite C-160) sodium cation exchange resin can be placed into 50 CC of DI water containing 5 g of AgNO₃ and the mixture stirred for 15 hours in an Erlenmeyer flask covered with aluminum foil. The resin can be filtered and washed repeatedly with DI water to yield the Amberlite —SO₃⁻ Ag⁺ resin. The 10% PSS solution (10 g) can be added to the Amberlite Ag⁺ resin and the dispersion slowly stirred overnight. The ion exchange resin can be filtered away and the water removed to yield a mixed Ag⁺, Na⁺ salt of sulfonated polystyrene.

### EXAMPLE 2

### Preparation of Material 18

Polystyrene sodium sulfonated (PSS, 70,000 mw) acquired from Sigma-Aldrich, PO Box 14508, St. Louis, Mo. 63178, UNITED STATES can be dissolved into DI water to yield a 10% solids solution. Separately, 25 g of Amberlite (Purolite C-160) cation exchange resin can be placed into 50 CC of DI water and 10 CC of 12N HCl can be added, and the resin stirred for 2 hours at room temperature. The resin can be filtered, washed extensively with DI water and a solution of arginine:HCl in DI water (10 mg/mL) added, and the solution stirred for 10 hours at room temperature. The resin can be filtered and washed with warm DI water and 100 g of the PSS solution added and the mixture stirred overnight in an Erlenmeyer flask covered with aluminum foil. The ion exchange resin can be filtered away and the water removed to yield a mixed arginine⁺, Na⁺ salt of sulfonated polystyrene.

### EXAMPLE 3

### Preparation of Material 18

Polystyrene sodium sulfonated (PSS, 70,000 mw) acquired from Sigma-Aldrich, PO Box 14508, St. Louis, Mo. 63178, UNITED STATES can be dissolved into DI water to yield a 10% solids solution. Separately, 25 g of Amberlite (Purolite C-160) cation exchange resin can be placed into 50 CC of DI water and a 20 CC of a solution of doxycycline:HCl in DI water (10 mg/mL) can be added and stirred for 15 hours. The resin can be filtered, washed extensively with Di water dried to yield the Amberlite —SO₃⁻ arginine:H⁺ resin. The 10% PSS solution (100 g) can be added to the Amberlite doxycycline:H⁺ resin and the dispersion slowly stirred overnight. The ion exchange resin can be filtered away and the water removed to yield a yellow mixed doxycycline⁺, Na⁺ salt of sulfonated polystyrene.

### EXAMPLE 4

### Application of Material 18

PSS (70,000 molecular weight, 5 g) can be combined with 45 g of hydrophilic polyurethane (PSS formulation) and the mixture dissolved into a 95:5 mixture of ethanol-DI water to yield a 10% solids solution. The solution can be cast in to a film, air dried and vacuum dried to yield a flexible material. The polymer-PSS formulation can be used to evaluate the effect of PSS against elastase. The results are detailed below. Note that the PSS formulation (blue bar) has reduced the elastase from 30 milliunits to approximately 6 milliunits reflecting a roughly 80% decrease in activity as depicted in the graph below.

### EXAMPLE 5

### Application of Material 18

About 15 g of Cutinova amorphous hydrogel (Beiersdorf AG, Unnastraβe 48, D-20245, Hamburg, Germany) can be transferred to a vial and 1.67 g of PSS (mw=70,000) (Sigma-Aldrich, PO Box 14508, St. Louis, Mo. 63178, UNITED STATES) added, and the PSS stirred into the gel using a glass rod to yield about a 10% (wt./wt.) solids composition. The data are presented for PSS alone (70K and 1000K molecular weights) as well as formulation 188-DJV. The bar graph PSS-formulation reveals that nearly 80% of the elastase is removed from the test sample (human wound fluid).

### EXAMPLE 6

### Application of Material 18

The sample from example 5 above (DJV-188) can be compared to Polystyrene sodium sulfonates (PSS 70K and 1000K) dissolved in buffer. These identifiers are molecular weights of these materials. These PSS-containing formulations can be compared to Cutinova gel (same as unlabeled gel), an ion-exchange polymer (nuggets), and gauze.

### EXAMPLE 7

### Application of Material 18

About 25 grams of sodium alginate (Sigma-Aldrich, PO Box 14508, St. Louis, Mo. 63178, UNITED STATES) can be combined 5 with 250 mL of sterile DI water (as by autoclave sterilization) and the mixture can be autoclaved in order to facilitate dissolution. Simultaneously 15 grams of PSS (1000K) can be combined with 200 mL of sterile DI water and autoclaved (to 105° C.) as the above described solution in order to facilitate dissolution. Following autoclaving the above solutions can be combined and filtered through polyester fabric in order to remove non-dissolved matter. The combined solution can be autoclaved one more time (105° C.) and capped in order to ensure shelf life. Separately, 1 liter of 0.5M CaCl₂ can be prepared and 10 grams of PSS (1000K) added in order to ensure that little 15 PSS is lost in the crosslinking step. The alginate solution can be added dropwise to the calcium chloride PSS solution in order to prepare beads. The beads can be allowed to dwell in the calcium chloride solution for 5 minutes and subsequently filtered through polyester fabric. The beads can be packaged and refrigerated prior to testing.

With the alginate-PSS solution from above, a sheet of Evolon 130 (gr) soft (Freudenberg/Evolon NA) can be immersed so as to become fully wetted and the fabric removed and excess alginate solution removed. The fabric can be placed into the CaCl₂-PSS solution and allowed to dwell until the alginate has become firm. The fabric composite can be cut to size and sterilized by electron beam irradiation (25 kG) prior to studies.

### EXAMPLE 8

### PVA-PSS Composite

Polyvinyl alcohol (PVA, PVOH) is a polymer that can be prepared by the (partial or complete) hydrolysis of polyvinyl acetate. The polymer can be water soluble, non-toxic, and hydrophilic leading to its hydrogel characteristics. To prepare PVA hydrogel samples incorporating PSS, a PVA/DI H₂O mixture can be first formed based on the desired weight percentage (15% solids). The mixture can be autoclaved on a liquid cycle with a chamber temperature of 121° C. and a sterilization time of 30 minutes in order to form a homogeneous solution. Similarly, PSS (average MW=70,000 or 1,000,000) can be suspended in DI H₂O at a proportion to yield a 10% solids solution and the mixture can be autoclaved on a liquid cycle with a chamber temperature of 121° C. and a sterilization time of 30 minutes in order to form a homogeneous solution. The solution can be allowed to cool and the PSS solution combined with the PVA solution in the ratio of 20:80 to yield a composite which is 14.2% PSS (dry weight). The solution can be blended to homogeneity and cast into a rectangular mold at an elevated temperature (>100° C.) and treated with a freeze/thaw cycling scheme. A single cycle refers to 8 hours of freezing at −20.0° C. and 4 hours of thawing at 22° C. All cycles can be applied sequentially for the desired number of repetitions. Following cycling completion, the rectangular specimens can be carefully die cut into the desired shape.

The PSS-containing beads can be effective at inhibiting elastase (see bar chart right) IMS-70-1, IMS-70-2, IMS-70-alginate, and IMS-1000-aliginate. Similarly the products can be effective against Cathepsin G, MMP-8, and MMP-9.

### EXAMPLE 9

### PSS Derivitization with Aarginine

Polystyrene sulfonate (Aldrich Chemical, 434574) average Mw=1,000,000 can be dissolved as described in the previous example to yield a 20% solids solution in Di water. 100 grams of The solution (100 grams) can be placed into a SnakeSkin® Dialysis Tubing (available from Pierce, P.O. Box 117, Rockford, Ill. 61105), 10K MWCO and the tubing clamped. The balloon of the tubing can be placed into a DI water bath (2 L) containing 25 grams of arginine-HCl (Sigma). The balloon can be allowed to equilibrate for 24 hours, removed, rinsed in DI water and then placed into a DI water (only) bath and changed every 4 hours for a total period of 72 hours. The resulting solution can be lyophilized to yield a flocculent hydroscopic solid. The composition of this example can be effective at inhibiting Serine Proteases, MMP's, Elastase, and/or Cathepsin G, for example.

1. A method for promoting healing of tissue of a multicellular organism, comprising administering a therapeutically effective amount of a polysulfonated material in a liquid mixture to reduce one or both of inflammation and cancerous cell growth.

2. (canceled)

3. (canceled)

4. (canceled)

5. The method of claim 1 wherein the administrating comprises applying the polysulfonated material in the liquid mixture to an epithelial, dermal, epidermal, subdermal, or periodontal wound.

6. (canceled)

7. (canceled)

8. The method of claim 5 wherein the wound is a burn, chronic wound, or inflammatory skin disorder.

9. (canceled)

10. The method of claim 8 wherein the chronic wound is one or more of a pressure ulcer, a diabetic ulcer, a venous leg ulcer, and/or periodontitis.

11. The method of claim 5 wherein the administering comprises applying the polysulfonated material in the liquid mixture topically to the epithelial, dermal, epidermal, subdermal, or periodontal wound.

12. The method of claim 1 further comprising inhibiting one or more gelatinolytic, elastolytic, or collagenolytic proteases and subsequent protein degradation of the organism after administering the mixture.

13. (canceled)

14. (canceled)

15. (canceled)

16. (canceled)

17. (canceled)

18. (canceled)

19. (canceled)

20. (canceled)

21. (canceled)

22. The method of claim 1 wherein further comprising associating the polysulfonated material with a solid material prior to the administering.

23. (canceled)

24. The method of claim 1 wherein the administering further comprises providing at least one additional biologically active material.

25. (canceled)

26. The method of claim 24 wherein the biologically active material comprises one or more of a peptide, a protein, a cytokine, a healing factor, an antibiotic, a biological process inhibitor, and a cytotoxin.

27. A composition for healing the tissue of a multicellular organism, comprising a polysulfonated material in a liquid mixture, the composition configured to be administered to reduce one or both of inflammation and cancerous cell growth.

28. The composition of claim 27 wherein the sulfonated material comprises a polymer having at least one sulfonate group extending from the molecular backbone.

29. (canceled)

30. The composition of claim 27 wherein the sulfonated material comprises at least one terminal sulfonate group and the sulfonate group is associated with a nitrogen containing group of an organic or organic metallic compound.

31. (canceled)

32. (canceled)

33. (canceled)

34. The composition of claim 27 wherein the sulfonated material is associated with an organic cation, the organic cation being one or more of a tetracycline, doxycycline, arginine, lysine, glutathione, mafenide, albuterol, and lidocaine.

35. The composition of claim 27 wherein the sulfonated material comprises one or more monomer of an arylenevinyl sulfonate, styrene sulfonate, vinyl sulfonate, and sulfated saccharide.

36. (canceled)

37. (canceled)

38. (canceled)

39. (canceled)

40. (canceled)

41. (canceled)

42. The composition of claim 27 wherein the liquid mixture is one of a gel, paste, cream, or lotion.

43. (canceled)

44. (canceled)

45. (canceled)

46. (canceled)

47. The composition of claim 27 wherein the liquid mixture is a hydrogel formulation, the sulfonated material being blended into the hydrogel formulation.

48. The composition of claim 47 wherein the hydrogel formulation comprises one or more of a polysaccharide, polyacrylate, polyalkylene oxide, poly(N-vinyl pyrrolidone) and a polyurethane.

49. (canceled)

50. The composition of claim 27 wherein the mixture comprises one or more of a viscosity modifier, lubricant, detergent, a petrolatum, an excipient, a wetting agent, and a skin permeation enhancer.

51. (canceled)

52. (canceled)

53. A composition for healing the tissue of a multicellular organism, comprising one or both of a biodegradable or nonbiodebradable solid material, the particles comprising at least one polysulfonated material, and configured to be administered to reduce one or both of inflammation and cancerous cell growth.

54. (canceled)

55. (canceled)

56. (canceled)

57. (canceled)

58. (canceled)

59. (canceled)

60. (canceled)

61. (canceled)

62. (canceled)

63. The method of claim 24 wherein the biologically active material is at least one of an antimicrobial agent, angiogenic agent, antifungal agent, antiviral agent, antiparasitic agent, anesthetic, enzyme, enzyme inhibitor, growth factor, anti-inflammatory agent, antihistamine, analgesic, antineoplastic agent, hormone, cytotoxic agent, tranquilizer, metal ion, vitamin, amino acid, nucleic acid, protein, peptide, cytokine, monoclonal antibody, and antiangiogenic agent.
